(19) United States
(12) Patent Application Publication
(10) Pub. No.: US 2023/0043662 A1
(43) Pub. Date: Feb. 9, 2023

(54) METHOD OF PRODUCING A COMPOSITION FOR THE TREATMENT OF CARDIOVASCULAR AND CEREBROVASCULAR DISEASES

(71) Applicants: Aslan Yavuz HABERDAR, Atasehir/Istanbul (TR); Vladislav LASKAVYI, Saratov (RU)

(72) Inventor: Aslan Yavuz HABERDAR, Istanbul (TR)

(73) Assignees: Aslan Yavuz HABERDAR, Istanbul (TR); Vladislav LASKAVYI, Saratov (RU)

(21) Appl. No.: 17/793,428

(22) PCT Filed: Dec. 30, 2020
(86) PCT No.: PCT/TR2020/051457

§ 371 Date: Jul. 18, 2022

(30) Foreign Application Priority Data
Jan. 17, 2020 (TR) 2020/00720

(51) Int. Cl. A61K 31/115; A61K 9/08; A61K 47/02

(52) U.S. Cl. CPC A61K 31/115; A61K 9/08; A61K 47/02

(57) ABSTRACT

A method of producing a composition for a treatment of cardiovascular and cerebrovascular diseases is provided. The method includes the following steps of: a) according to a ratio of 1 lt, adding 2 ml 36.5-37.5% formic aldehyde solution to 998 ml sterile and 0.85-0.95% isotonic sodium chloride solution to obtain a mixture, mixing the mixture thoroughly and obtaining an aldehyde solution with a final concentration of 0.073-0.075% by weight for injection, and b) storing the composition obtained in a dark place at a temperature of 15-35° C.

### CROSS REFERENCE TO THE RELATED APPLICATIONS

This application is the national stage entry of International Application No. PCT/TR2020/051457, filed on Dec. 30, 2020, which is based upon and claims priority to Turkish Patent Application No. 2020/00720 filed on Jan. 17, 2020, the entire contents of which are incorporated herein by reference.

### TECHNICAL FIELD

The invention relates to a method of producing a composition for the treatment of cardiovascular and cerebrovascular diseases.

### BACKGROUND

Cardiovascular and cerebrovascular diseases are the main diseases that threaten people's life and health. These diseases have high recurrence and high mortality rates.

Cardiovascular and cerebrovascular diseases trigger coronary heart disease, angina pectoris, myocardial infarction, ischemic encephalopathy, cerebral thrombosis, hypertension, vascular dementia, atherosclerosis of cerebral and cervical arteries and similar diseases. The main causes of these diseases are vascular stenosis and obstruction of the ducts leading to insufficient blood flow to the heart and brain. It causes a feeling of heaviness, dizziness, headache, chest tightness and other symptoms. One common type of cerebrovascular disease is vascular dementia. High blood pressure, single or repeated insult (stroke), high cholesterol, diabetes, smoking, alcohol consumption, etc. causes trigger the disease. It manifests itself in memory impairment, counting ability disorder, disorientation, loss of thinking ability, and even life loss when it reaches an advanced stage.

A wide variety of methods are known in the art for treating cardiovascular diseases. Common of these methods; glycosides, vasoconstrictors, vasodilators, cardio protective drugs.

The document numbered RU2446806 can be shown as an example of the state of the art in the research conducted in the literature. The document relates to a cardioprotective, antiarrhythmic, anti-ischemic drug for the treatment of various forms of coronary heart disease containing acetylsalicylic acid I, L-arginine hydrochloride and folic acid and used in tablet form. However, the scope of treatment of said drug is narrow. Because it cannot be used to treat cerebrovascular diseases. There are compositions known in the art for the treatment of cardiovascular and cerebrovascular diseases.

The document numbered WO2019196835 can be shown as another example of the state of the art. The document relates to a composition for treating ischemic stroke, cerebral infarction, cerebral stroke, and cerebral atherosclerosis. R1, R2, R3, and R4 are independently selected from O(CH2)nCH3, halogen atom, H, CF3, OCF3, OH, SH, NH2, carboxyl, ester, sulfone, sulfoxide, sulfonate, sulfonate Ester group, sulfonamide group, ketone group, aldehyde group, nitro group, nitroso group, C1-12 hydrocarbon group and C1-12 aryl group. n is 0 or a positive integer. R5 is H, CF3, OCF3, OH, C1-12 hydrocarbon group or C1-12 aryl group. R6, R7, and R8 are each independently selected from halogen atoms, H, CF3, OCF3, OH, SH, NH2, carboxyl, ester, sulfone, sulfoxide, sulfonate, sulfonate, sulfonamide, Ketone, aldehyde, nitro, nitroso, C1-12 hydrocarbon group and C1-12 aryl group. X is O or NH. Y represents R9 selected from the side chain of any of the natural amino acids that do not contain proline. R10 represents H, carboxyl, amide or ester.

The document numbered RU2012134284 can be shown as another example of the state of the art. Said document is a drug for the treatment of cardiovascular diseases containing dihydropyrazolone derivatives. The compounds described in the present invention are very complex in their composition, containing a number of ingredients that cause side effects.

Another example is document number RU2674264. The drug for the treatment of cardiovascular and cerebrovascular diseases contains 1 part ginseng, 0.8-1.5 parts ginkgo leaf, 0.018-0.030 part saffron stamp. The composition of the present invention can be used in solid, liquid or gas forms. When used in solid form, the composition can be in the form of tablets, powder, granules, capsules. When used in liquid form, the composition can be in the form of an oral liquid, suspension, syrup, tincture, injection. The gaseous composition can be used as an aerosol or inhalation. This composition can be used in the treatment of ischemic cerebrovascular disease, coronary heart disease, angina pectoris and vascular dementia. Doses of the composition prepared from ginseng, ginkgo leaves and saffron droplets to reduce side effects caused by long-term use can be reduced. The drug contains ginseng as a biologically active supplement, which can lead to unpredictable results. Medicines based on herbal ingredients generally cause severe allergic reactions.

The natural metabolite formic acid aldehyde is known to be used in various disorders of the body's immune system, immunocorrection, infectious diseases and oncology. It is also used in the treatment of viral infections, as a cholesterol regulating component, to activate the body's own stem cells. However, the use of a formic aldehyde-based drug for the treatment of cardiovascular and cerebrovascular diseases is not known to date.

As a result, the existence of the above problems and insufficiency of existing solutions made it necessary to make an improvement in the related technical field.

### SUMMARY

The present invention relates to a method of producing a composition for the treatment of cardiovascular and cerebrovascular diseases, which overcomes the aforementioned disadvantages and brings new advantages to the related technical field.

The main aim of the invention is to obtain a composition containing aqueous formic aldehyde solution.

The aim of the invention is to activate the natural intracellular metabolite activation and proliferation in the human body, which makes the treatment of cardiovascular and cerebrovascular diseases more effective.

Another object of the invention is to increase the efficacy of treating cardiovascular and cerebrovascular diseases.

Another object of the invention is to provide a non-toxic and anti-acclimatization component for the treatment of cardiovascular and cerebrovascular diseases.

### DETAILED DESCRIPTION OF THE EMBODIMENTS

In this detailed description, preferred alternatives to the composition production method for the treatment of cardiovascular and cerebrovascular diseases according to the invention are described solely for a better understanding of the subject and without any limiting effect.

The production method of the composition of the invention is as follows;
a) 2 ml 36.5-37.5% formic aldehyde solution, 998 ml sterile 0.85-0.95% sodium chloride solution, 0.073-0.075% aldehyde solution for injection are obtained according to 1 lt ratio.
b) The composition obtained is stored in a dark place at a temperature of 15-35° C.

For example; 0.2 ml of 37% formic aldehyde solution is added to 998 ml of sterile and 0.90% or 0.95% isotonic sodium chloride solution to thoroughly mix the mixture to obtain a final concentration of 0.074% by weight formic aldehyde.

As another example; 0.2 ml of 36.5% formic aldehyde solution is added to 998 ml of sterile and 0.90% or 0.95% isotonic sodium chloride solution to thoroughly mix the mixture to obtain a final concentration of 0.073% by weight formic aldehyde.

Aqueous formic aldehyde solution (formic acid aldehyde) is a clear, colorless liquid with a distinct pungent odor that can be mixed with water and alcohol in all proportions. Formic aldehyde is a representative of the HCHO aldehyde class. It is a colorless gas with a pungent odor. It weighs 30.03, its density at −20° C. is 0.815 g/cm3, its melting point is −92° C. and its boiling point is −19.2° C. It is soluble in water and alcohol.

Isotonic sodium chloride solution is a colorless transparent liquid with salt. The solution is sterile, pyrogen-free.

Sodium chloride, cubic crystals or white crystalline powder, salty tasteless and odorless. It is soluble in water.

The aqueous formic aldehyde solution obtained with the composition of the invention is a clear, colorless, odorless liquid with a slightly salty taste.

The aqueous formic aldehyde solution obtained with the composition of the invention is administered orally and/or intramuscularly.

5 ml is administered by injection once a day on days 1, 3, 7, 10, 14.

When administered orally, the composition is drunk for 10 ml in the morning and evening on an empty stomach 1/1.5 hours before or after a meal.

The composition of the invention is applied for 5, 7 or 10 days. It can be repeated 10, 20 or 30 days after the last application. The repetition of oral and/or intramuscular administration is decided according to the patient's clinical and biochemical parameters, taking into account the physiological condition.

In the injection of formic aldehyde into the body at the determined concentration ratio, the level of changes in the rate of glycolysis in the cytoplasm of the cells and the normalization of the physiological state of the cardiovascular system cells by aerobic oxidation related to mitochondrial activity were achieved.

What is claimed is:

1. A method of producing a composition for a treatment of cardiovascular and cerebrovascular diseases, comprising the following steps of:
a) according to a ratio of 1 lt, adding 2 ml 36.5-37.5% formic aldehyde solution to 998 ml sterile and 0.85-0.95% isotonic sodium chloride solution to obtain a mixture, mixing the mixture thoroughly and obtaining an aldehyde solution with a final concentration of 0.073-0.075% by weight for injection; and
b) storing the composition obtained in a dark place at a temperature of 15-35° C.

2. The method according to claim 1, wherein the composition comprises an oral and/or intramuscular administration of an obtained aqueous formic aldehyde solution.
